(19) United States
(12) Patent Application Publication
(10) Pub. No.: US 2006/0015042 A1
(43) Pub. Date: Jan. 19, 2006

(54) Method and apparatus for detection and measurement of scoliosis of the spine

(76) Inventors: Andrew V. Linial, Stuart, FL (US); Robert W. Atkinson, Daytona Beach, FL (US); Richard Ejgird, Stuart, FL (US)

Correspondence Address: SHERMAN & ASSOCIATES, 415 NORTH ALFRED STREET, ALEXANDRIA, VA, 22314, US

(21) Appl. No.: 10/891,001

(22) Filed: Jul. 15, 2004

(51) Int. Cl. A61B 5/103

(52) U.S. Cl. 600/594

(57) ABSTRACT

The present invention is directed to a method and apparatus for measuring the lateral curvature of the human spine to indicate the presence and degree of scoliosis and is useable by non-medical personnel and medical practitioners, including doctors, chiropractors, physical therapists, nurses and the like. The system includes a low cost quick operating hand held device based on the operating structure of a standard computer mouse to immediately determine the curvature of the spine. The hand held device scans the length of a patient's spine by contacting the patient's back as it is drawn along the length of the spine to provide a reading of the lateral curvature as X-Y coordinate information which is then graphed by a computer software program which can be run on a standard PC of the desktop, laptop, tablet or other type. The software program allows repeated readings to be saved and compared thereby providing a patient history over time.

### FIELD OF THE INVENTION

The present invention is directed to a method and apparatus for detection and measurement of scoliosis of the spine which is non-invasive and does not involve X-ray analysis. The invention provides a system for measuring the lateral curvature of the human spine to indicate the presence and degree of scoliosis and is useable by non-medical personnel and medical practitioners, including doctors, chiropractors, physical therapists, nurses and the like. The system includes a low cost quick operating hand held device based on the operating structure of a standard computer mouse to immediately determine the curvature of the spine. The hand held device scans the length of a patient's spine by contacting the patient's back as it is drawn along the length of the spine to provide a reading of the lateral curvature as X-Y coordinate information which is then graphed by a computer software program which can be run on a standard PC of the desktop, laptop, tablet or other type. The software program allows repeated readings to be saved and compared thereby providing a patient history over time.

### BACKGROUND OF THE INVENTION

Scoliosis is a three dimensional deformity of the spine in which there is one or more lateral curvatures away from the vertical in the thoracic or lumbar portion of the spine. It may develop as a single curve resembling the letter C or as two curves, as in the letter S, mostly in the thoracolumbar region of the spine. Although in many cases, a small amount of curvature does not worsen and is hardly noticeable, this curvature may result in poor cosmetic appearance due to a tilted body position, or in extreme conditions it can severely impact a person's ability to function normally. The severity of scoliosis is diagnosed according to the curve and by the angle of trunk rotation. The two factors are usually related such that, for example, one may have a curve of 20 degrees and a trunk rotation of 5 degrees. Scoliosis is diagnosed when the curve measures in excess of 10 degrees, but treatment is not usually required until the curve reaches 25 degrees with a rotation of more than 7 degrees. Mild scoliosis, defined as a curvature of less than 20 degrees, is not serious and requires only monitoring every 6 months.

While scoliosis can be found in infants through adults, it is primarily initiated when a child enters puberty. Therefore, schools presently check for this disease in the fifth to seventh grade through an examination by a medically trained individual. Simple visual screening will spot most cases, although there is no systematic method of noting the change in spine development through the critical teenage growth years.

Scoliosis is more prevalent in females than males and is most often seen in ages 10-16. The American Academy of Pediatrics recommends that girls be screened at least twice, at ages 10 and 12, and boys at least once about the age of 13 or 14. Older teenagers, 11^{th} and 12^{th} grades, should be given additional screenings since about 40% of those screened earlier have scoliosis that develops later in the teenage years or were undetected in earlier screenings. The recommendation of the American Academy of Pediatrics is therefore to screen children at the ages of 10, 12, 14 and 16. However, the U.S. Preventive service Task Force has recommended against routine screening due to inaccuracies of the screening tests and the dependence on the skill of the examiner. Students with minor curvature who are not at risk for progression are often referred to a physician by overcautious schools which only adds to the cost of the health care system. Since early detection is based on a visual examination of the patient in two positions, standing and bent over, the appearance of a minor curve in a child's spine is reported to the school and the child is referred to a physician for more detailed examination using the X-ray method.

The most effective and accurate understanding of the curvature of the spine is obtained through a series of X-rays and careful measurement of the spine directly on the developed film by a qualified radiologist. However, such procedures are time consuming and expose the patient to repeated doses of radiation. Scoliosis is determined by a measurement of the included angle, known as the Cobb angle, of the ends of the curve and is generally diagnosed when this angle exceeds 10 degrees. The angle is usually measured through the use of X-rays and plotting of the angle directly on the developed film by means of structural lines, and measurement with a two armed goniometer. This process requires a degree of knowledge and skill on the part of the technician. In addition, over exposure or under exposure of the X-ray film can make it difficult to accurately plot the angle resulting in potentially significant errors and inaccurate reporting.

There are also contact devices available for scanning the back and reporting on the position of the vertebrae. Such devices tend to be either overly simple and rely on the user to take hand written notes during use, or they are large and expensive and not readily transportable.

For example, U.S. Pat. No. 5,181,525, Bunnell, presents a scoliosis screening device in the form of an inclinometer having a U-tube filled with fluid to dampen the motion of a ball within the tube which seeks the lowest point in the tube. The tube is encased in a plate having an indentation in its bottom edge which conforms to a patient's back and spinal column, the plate also having indicia thereon adjacent to the tube representing degrees of rotation.

Measurement of a patient's back is taken by having the patient bend forward to expose the point of maximum deformity, which should be viewed at eye level from behind the patient. The device is laid across the apex of the deformity, perpendicular to the long axis of the body, and the angle of inclination is read directly from the device. Placement of the device depends on the curve pattern and data is manually recorded on a clinical summary sheet. With this device, a complete image of the spine is only obtained by painstaking plotting of the summary sheet data on a graph representing the patient's spine. In addition, the device requires multiple placements and recordings to chart the patient's spine.

U.S. Pat. No. 6,468,233, Cook, presents a posture analyzer for analyzing a patient's spine and comprising an upright standard secured to a base resting on the floor. A rectangular frame has one side frame member secured to the upright standard at a selected height with the frame member supporting a markable panel on which is provided an orthogonal grid pattern and a depiction of a normal spine. A bar holder is slidably secured to a second side frame and is movable in the vertical direction along the second side frame member. The bar holder slidably holds a bar which extends in a horizontal direction parallel to the planar surface of the panel and has a roller secured to one end for rolling up and down a patient's spine and a pen at the opposite end for marking on the planar surface of the panel. When the roller is moved vertically and horizontally along a patient's spine, the pen makes a corresponding tracking mark on the panel.

The device of this patent is limited to use on a patient standing in a vertical position and produces a representation of the spine that is limited to the dorso-ventral direction. It cannot produce data representing lateral curvature. As such this device is of limited utility in diagnosing and measuring scoliosis.

U.S. Pat. No. 4,723,557, Gross, presents a lordosimeter which employs point encodement and codement of spatial measurement of spinal landmarks to provide data for a three dimensional representation of the configuration of the spine for diagnosis and assessment of lordosis and scoliosis. The device comprises a telescopic steel rod mounted in a fixed housing and extendable therefrom. The rod is fitted at its distal end with a body contacting point and is connected at its proximate end to the vertexes of two orthogonally mounted linear potentiometers. Displacement of the orthogonal mounting provides analog signals generated in the potentiometers indicating azimuth and elevation. A third linear potentiometer mounted in the housing is connected to the extensible end of the telescopic rod and provides an analog signal indicating extension as the rod is extended and retracted. The analog signal outputs of each of the potentiometers are linearly amplified to the range of measurements of spinal positional parameters with the analog signals representing elevation, azimuth and extension with respect to the fixed position of the device and the measured spinal points.

The Gross device is not readily portable as the position of the device must be fixed relative to the position of the patient. In addition, the device requires care in placement of the locating pointer on the end of the telescopic rod at each landmark point to be plotted on the patient's back.

U.S. Pat. No. 6,637,278, Fasanella, presents a measuring system for determining the surface line of a body which employs two acceleration sensors in a common plane but disposed at right angles to one another. Acceleration sensors generate signals upon changes of the position of the measuring axes with respect to the axis of acceleration due to gravity. A single measuring wheel provides a linear measurement component. Signals from the acceleration sensors and the measuring wheel are transferred as data to a computer wherein angles of inclination are determined at predetermined path and/or time intervals and the data belonging to specific measuring points is calculated and used to chart the course of the device.

Because the Fasanella device uses only a single measuring wheel, the device is susceptible to inadvertent sideways or fore and aft tilting by the operator during use. Since the acceleration sensors generate their signals in response to their relative position with respect to gravity rather than the actual position of the device on the body being measured, any tilting by the operator during use will be incorporated into the signal produced by the acceleration sensors with the result that the measurement will contain errors that are not representative of the actual physical condition being measured.

Proper diagnosis is important since a misjudgment can lead to unnecessary use of X-rays and unnecessary fear in children and their parents where there is no risk of actual curvature progression. The present invention seeks to overcome the deficiencies of the prior art by providing a compact, inexpensive, easy to use device that uses a familiar input means to obtain a direct and accurate measurement of a person's spine without resorting to the use of X-rays. By providing a readily portable, inexpensive, easy to use, non-invasive tool multiple screenings may be made of patients over time and the results stored and compared to readily and quickly determine if there is any progression of the condition to warrant reference to a physician.

### SUMMARY OF THE INVENTION

It is therefore an object of the present invention to provide a method and apparatus whereby accurate meaasurements of a person's spine can be obtained without resorting to the use of X-rays.

It is a further object to provide a method and apparatus for determining scoliosis of the spine that is simple and easy to use.

It is an even further object to provide an apparatus and method for determining and measuring the degree of scoliosis of the spine that is portable and non-invasive.

Further objects and advantages will become evident from the following description which provides an apparatus and method for the detection and measurement of spinal abnormalities where the apparatus comprises a hand held data input means having a track ball mechanism adapted for linear translation along a person's spine and adapted to generate data signals corresponding to direction and distance of motion of the rack ball in X and Y coordinate planes, a computer, a data transmission connection between the hand held input means and the computer to transmit data signals from the data input means to the computer, and a software program running on the computer that is adapted to receive the data transmission from the data input means and convert the data signals into a visual output representative of a patient's spine and to calculate the Cobb angle and spinal deviation representative of scoliosis.

The present invention further provides an apparatus for detection and measurement of spinal abnormalities comprising a hand held data input means having a track ball mechanism adapted for linear translation along a patient's spine and adapted to generate data signals corresponding to direction and distance of motion of the track ball mechanism. The hand held data input means comprises a housing having an upper surface contoured to fit a human hand and a lower surface of substantially planar dimension, the housing containing a track ball mechanism comprising a ball rotatably confined therein so as to partially protrude from an aperture in the lower surface whereby the ball is capable of contacting and rolling along a surface with the housing. An X-axis roller and a Y-axis roller are in contact with the ball within the housing and orthognal to each other with an X-axis pulse generator activated by the X-axis roller and a Y-axis pulse generator activatd by the Y-axis roller. The X-axis pulse generator and the Y-axis pulse generator each generate electronic signals in response to movement of the X-axis roller and the Y-axis roller by the ball and an electronic controller means receives and formats the signals into coordinate data. The housing further comprises a guide means on the lower surface in substantially linear relationship with the ball and a longitudinal axis of the housing. The guide means is adapted to engage on either side of the spine and thereby guide the hand held data input means along the spine. At least one control switch is provided on the upper surface of the housing and is electrically connected to the electronic controller and is actuatable during use of the hand held data input means to activate the controller and the pulse generators.

A method for scanning a spine, determining the presence of scoliosis and measuring deviation thereof with the apparatus is provided which comprises providing a hand held data input means and a computer, the hand held data input means comprising a track ball mechanism adapted for linear translation along a patient's spine where the track ball mechanism has means for generating data signals corresponding to direction and distance of motion of the track ball as X and Y coordinates and transmitting those data signals to the computer. Control means for activating the hand held data input means and a software program loaded on the computer and adapted to receive the data signals, process the data signals and generate a visual representation of the spine based on the data signals are also included. The software program includes means to enter personal data, create a file for maintaining the data and the visual representation as a data base file, calculate a Cobb angle from the data, and generate a report thereof

The method further comprises activating the software program and entering personal data for a patient, conducting a first scan by placing the hand held data input means on the patient's back such that the track ball is in contact with the back and against the spine at or about the T-1 vertebra, actuating the control means on the hand held data input means and moving the hand held data input means along the spine so that the track ball rolls along and follows the linear and lateral contours of the spine down to at or about the L-5 vertebra, whereby data signals are generated by the hand held data input means and are transmitted to the computer as a scan of the spine.

The signals are recorded and processed on the computer, the processing of the signals comprising generating a visual representation of the spine and calculating a Cobb angle therefor and generating a report following which the patient is diagnosed based on analysis of that report.

### BRIEF DESCRIPTION OF THE DRAWINGS

FIG. 1 illustrates the principle of a computer mouse.

FIG. 2 is an overhead view of a preferred embodiment of the hand held device of the present invention.

FIG. 3 is a rear view of a preferred embodiment of the hand held device of the present invention.

FIG. 4 is a side view of a preferred embodiment of the hand held device of the present invention.

FIG. 5 is a front view of a preferred embodiment of the hand held device of the present invention.

FIG. 6 shows a structure of the electronics of the hand held device of the present invention.

FIG. 7 is a sectional side view of a preferred embodiment of the hand held device of the present invention.

FIG. 8 shows the relationship of the hand held device to the computer.

FIG. 9 illustrates the operation of the hand held device on a patient.

FIG. 10 is a side view of the operation of FIG. 6.

FIG. 11 is a representative scan report as presented on the computer and in printed form.

### DETAILED DESCRIPTION OF THE INVENTION

The present invention makes use of the structure and operation of the common computer mouse to obtain a continuous measurement of the human spine.

Computer mice are commonly used pointing devices for personal computers (PCs) and are designed to inform PCs of movements in the X and Y coordinates and to initiate operations of computer software in response to activation of click switches provided on the outside of the mouse casing.

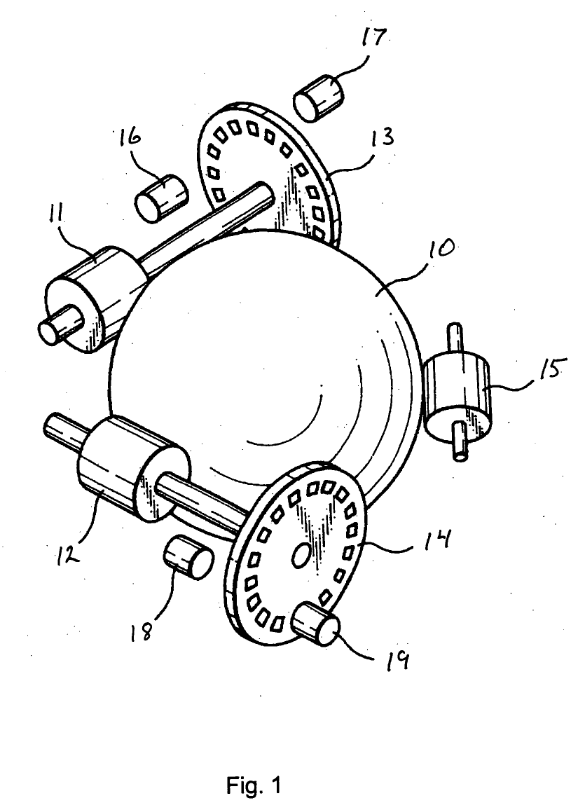

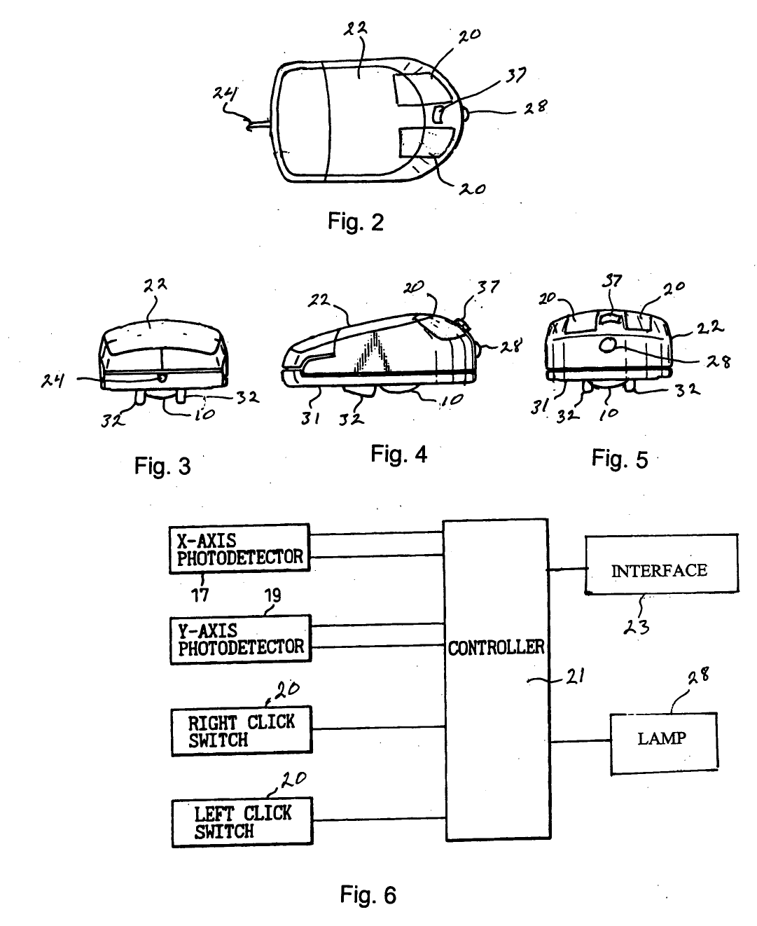

FIG. 1 shows the principle of a conventional mouse which is used in the present invention. A ball 10 turns in response to user's manipulation. The ball 10 is in contact with an X-axis roller 11 and a Y-axis roller 12 that are orthogonal to each other. The rollers 11 and 12 have disks 13 and 14, respectively. The disks 13 and 14 have slits formed at regular intervals and turn in respond to the rotation of the ball 10. A presser roller 15 is arranged at an angle of 45 degrees with respect to the rollers 11 and 12 to keep the ball 10 and rollers 11 and 12 touching each other.

An X-axis light emitter 16 and an X-axis photodetector 17 are arranged on the opposite sides of the disk 13, and a Y-axis light emitter 18 and a Y-axis photodetector 19 are arranged on opposite sides of the disk 14. The photodetectors 17 and 19 are connected to a controller 21, as shown in FIG. 6, which comprises a microprocessor to count the pulses from the photodetectors 17 and 19, monitor operations of the left and right click switches 20, format the pulse counts and click switch operations and transfer the formatted data to a PC.

A computer mouse detects its own movements along the X- and Y-axes and, through the pulses generated, produces corresponding movement of a cursor on a computer display. Operation of the click switches 20 on the mouse and the device of the present invention will result in various computer operations depending on the position of the cursor and the computer software then running.

The present invention makes use of the operation of a computer mouse type device to provide a method and apparatus for detection and measurement of scoliosis of the spine which is non-invasive and does not require exposure to X-ray or other radiation imaging means. As such, the apparatus is quick and easy to use and can be used on the same subject with greater frequency than X-ray analysis to monitor the presence and progression of scoliosis.

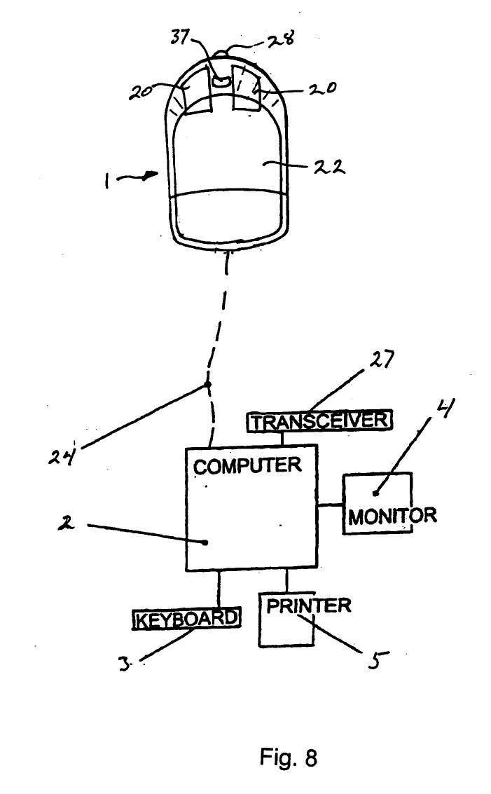

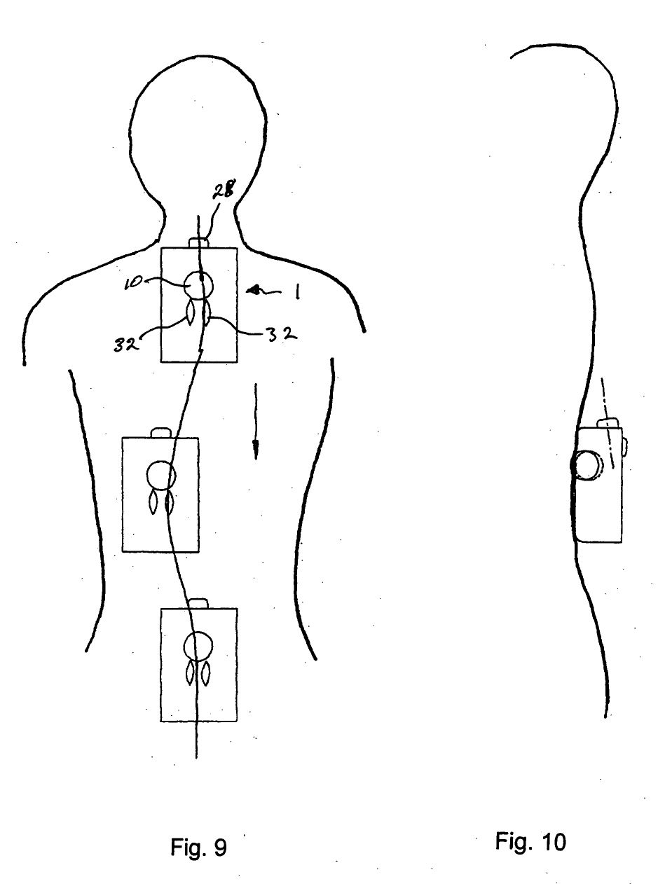

The apparatus is illustrated in FIG. 8 and comprises a hand held data input device 1 that is used to obtain a mechanical scan along the length of a subject's spine and provide X-Y coordinate data representative of the attitude of the spine to a computer 2. The computer includes a keyboard 3 for entering sata concernign the subject, a monitor 4 for displaying the results of a scan and a printer 5 for providing a hard record of the scan results. The computer 2 is preferably a standard PC and runs a software program written to accept the personal data concenring the subject and the X-Y coordinate data from the hand held device 1, process that data and generate a visual and printable report of the condition of the subject's spine based on that data.

The computer 2 with which the device and method of the present invention are used may be any PC whether desk top, lap top, tablet or other portable type. Preferably, a laptop or tablet PC is used. In addition, although the software program used with the device and method to generate scan reports from data obtained with the device has initially been written to run under Microsoft Windows operating system, it is within the scope of this invention to include its use and that of the device under other operating systems including, but not limited to, Linux and Apple.

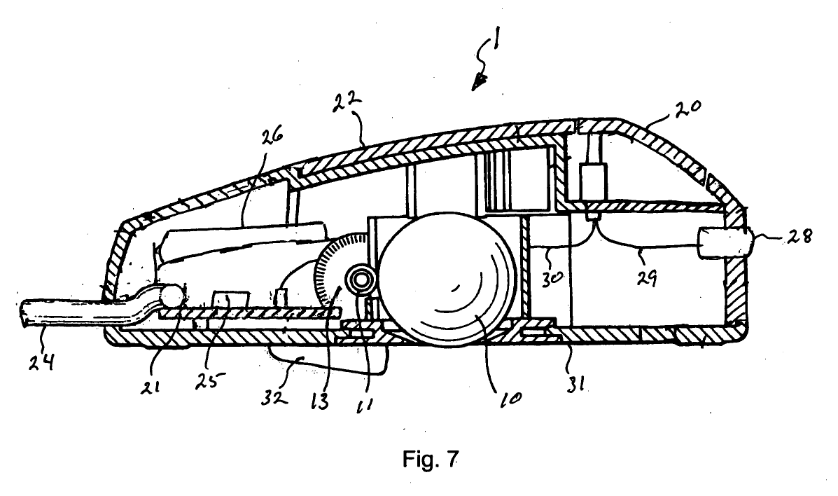

Referring to FIG. 7, the internal structure of the hand held device 1 is similar to a computer mouse except that the click switches 20 are at the end of the housing 22 opposite the cord 24. The photodetectors 17 and 19 are connected to a controller 21 within the housing 22 of the hand held input device which is preferably designed to fit comfortably and stably in the user's hand. The appearance of the housing 22 in the drawing figures is for illustration purposes only. Other housing designs may be used without departing from the scope of the present invention. Controller 21 receives pulses from the photodetectors 17 and 19 as well as on/off pulses from the click switches 20 and formats these pulses as data for transfer to the computer via a communication interface 23. This interface 23 may comprise a standard serial or USB cord 24 between the hand held device and the computer, or it may be a wireless communication interface such as infrared or RF such as are commonly used for short range communication between computers and peripherals. In such instance, the hand held device would include a transceiver 25 and a power source such as replaceable or rechargeable batteries 26 while the computer would be provided with a corresponding transceiver 27, either external or internal, for two way communication.

At the front of the housing 22, below the click switches 20, is a light source 28. Preferably an LED with a magnifier lens or a low power laser pointer lamp, the light source 28 is positioned to provide a spot beam on the subject's back at a point just forward of the hand held device 1. An angle of 30° to 50° for the beam relative to the plane of the underside 31 of the housing 22 is preferred. Light source 28 is preferably built into the structure of the hand held device 1 and is powered by the same power source. In that instance, light source 28 is activated by one of the click switches 20 via an electrical connection 29 and operation of the light source 28 in this manner also serves as an indication that the hand held device 1 is active and ready for use since the click switches 20 are also electrically connected to controller 21 via electrical connection 30. Alternatively, the light source 28 may be separately mounted on the hand held device 1 by a clip or receiving channel molded into the housing 22 in which case the light source 28 is removable and has its own power source and on/off switch.

On the underside 31 of housing 22 are guide members 32 which extend downward from the underside 31 of housing 22 behind and on either side of track ball 10. Guide members 32 are provided in at least two parallel, spaced apart pairs with one on either side of the longitudinal axis of the hand held device 1. The spacing between the guide members 32 is such that when the hand held device 1 is placed on a subject's back in line with the spine, the guide members 32 fit on either side of the vertebrae with the track ball 10 directly over the spine and in contact with the surface of the subject's back. In this manner, the guide members 32 provide lateral guidance as the hand held device 1 tracks along the spine. In their simplest form, the guide member 32 comprise at least one pair of skids molded as an integral part of the underside 31 of the housing 22 as shown in FIGS. 3-5. Although it is within the scope of this invention to have more than one pair of guide members 32, for example two pair, one ahead of and one behind the track ball 10, it is important that the guide members 32 not interfere with the operation of the track ball 10 or be so numerous or large as to restrict the ability of the hand held device to negotiate any curvature of the spine. The preferred embodiment is one pair of guide members 32 positioned as described and shown. As an alternative to the molded skids, guide members 32 may be provided in the form of freely rotating wheels extending through slots in the underside 31 of the housing 22, or as a series of separate short ridges in parallel spaced apart arrays along the underside 31 of the housing 22, or as the edges of a longitudinal channel molded in the otherwise planar underside 31 of the housing 22.

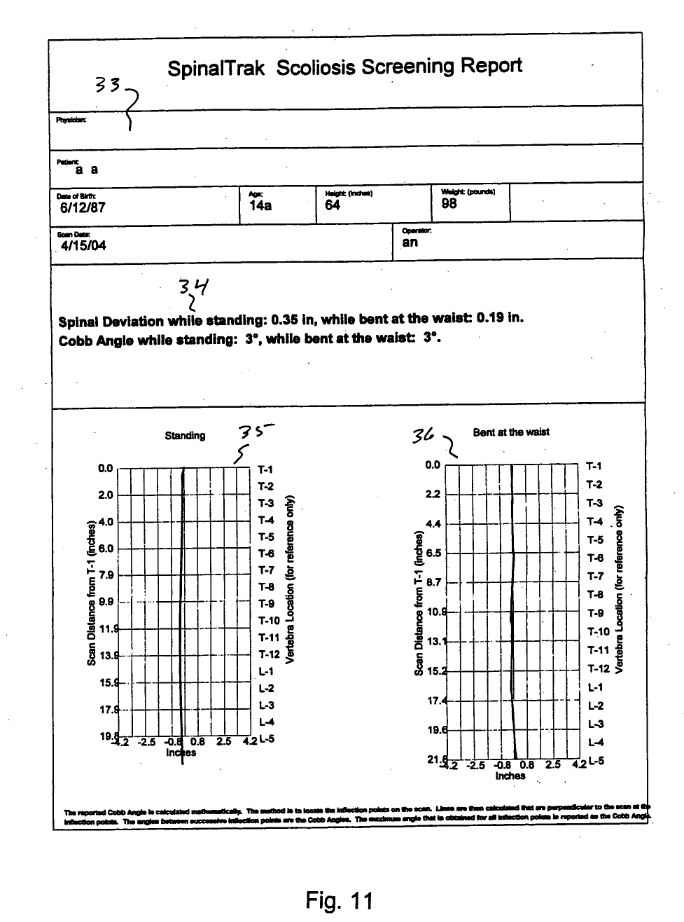

By means of the cord 24 or transceiver 25 or other interface 23 the hand held device 1 interfaces with the computer 2 on which a software program is running. This program takes the input signals from the from the hand held device 1 in the form of X and Y coordinate data, processes the data to minimize “noise” and smoothes the data into a continuous function. When two scans are performed, the final data is compared using a compare tolerance routine. If the horizontal difference between the two scans at every vertical point is less than the prescribed tolerance, the two scans are averaged together and displayed as a final scan, FIG. 11. If the difference between the scans exceeds the tolerance, the operator is prompted to repeat both scans.

Scans are conducted with the subject standing and with the subject bent over, and are created based on the distance from the T-1 vertebra to the L-5 vertebra. The locations of each vertebra are calculated assuming that the T-1 to L-5 distance is approximately equal to 31% of the subject's height when standing. When the subject is bent at the waist, the distance between the vertebrae naturally lengthens and is calculated to be 10% larger than when the subject is standing.

To create a scan using the apparatus and method of the present invention, an operator must first create or select a file in which the scan is to be stored. This is done on the computer 2 either by selecting an existing file or by initiating a new file which displays a dialog box on the monitor 4 in which the operator, using the keyboard 3 enters the relevant information about the subject to be scanned. Such information includes the subject's name, date of birth, age, weight, height, etc. This information is used to create a file in a database and allows the scan to be retrieved at a later date or a subsequent scan to be added and compared with previous scans. In addition, the entered height is used by the program to calculate the location of the vertebra from the T-1 to the L-5 on the basis that normal vertebra are evenly spaced and the distance form the T-1 to the L-5 accounts for 31% of a person's height on average.

Once the file is selected or created, the operator may initiate the scans using the hand held device 1 by drawing the device 1 downward along the spine from the T-1 to the L-5 vertebra while holding down a click switch 20 on the device 1 as shown in FIGS. 9 and 10. This is similar to the common “click and drag” operation of a computer mouse. The track ball 10 rolls along the spine generating X and Y coordinate data which is transmitted to the computer 2 and the program by the interface means 23. The guide members 32 on the underside 31 of the housing 22 are spaced apart so as to fit on either side of the spine thereby providing lateral guidance of the hand held device 1 along the spine. In addition, the light source 28 shining a spot on the subject's back as the device 1 is moved along assists the operator in following the geometry of the subject's spine. As an added feature, the device may include a bubble level 37 to assist the operator in maintaining the device 1 in the correct attitude thereby avoiding introduction of error due to inadvertent pitch and yaw movement of the device 1. Maintaining the hand held device 1 in an attitude that is perpendicular to the ground during a standing scan and parallel to the ground during a bent at the waist scan ensures accurate X and Y data is determined by the track ball 10 and communicated to the computer 2.

With the scans completed, they are then processed and compared to each other. The first part of this process is to align the scans which compensates for any differences in the actual starting location for each scan. In addition to alignment, processing the scans runs the data through the compare tolerance routine. The tolerances for comparison may be specified by the operator or they may be fixed. For example, in a version of the apparatus for standard use by school personnel, the tolerance may be fixed at 0.48-0.50 inches. Trained medical personnel, however, may wish to adjust the tolerances on the basis of a physical examination of the subject.

After the scans are processed, an average scan of each position is displayed on the monitor 4 and is automatically saved in the file. FIG. 11 shows a representative scan result as displayed. The results can be printed to provide a hard copy which can be carried to a physician if needed, or results can be transmitted electronically such as from a school to a physician's office over a network. To avoid inadvertent deletion of scanned data and a gap in the subject's record, it is preferred that there be no provision for removing a scan from a file once successful processing has been completed. If the program encounters an error at any time during processing, the processing of those scans is stopped and the operator provided with an error report and is prompted to repeat the scans. When this is done, the original scan data which generated the error report is deleted and is replaced with the new scan data for processing.

As part of the data processing, the program calculates the Cobb angle utilizing the X-Y coordinate input from the hand held device 1. First a curve is calculated that fits the input data. The program then calculates lines that are tangent to that curve every 0.25 cm. The angles of those lines are then used to locate the points of inflection of the curve and for each consecutive point of inflection, the absolute value of the angles are added together. This calculation is continued through all of the inflection points and the largest angle calculated is reported as the Cobb angle.

Referring now to FIG. 11, the basic scan report generated from the data received from the hand held device 1 is divided into three sections. First, the subject information 33 entered prior to the scan; second a notational report 34 generated by the program based on the data processed; and third, charts 35 and 36 of the actual scans. The subject information 33 identifies the subject of the scan and provides the particulars of age, height, weight, etc., the operator of the scan, the scan date and the physician, if any. The notational report is provided for scans in both positions and includes information relating to spinal deviation and the calculated Cobb angle. The operator or a reviewing physician may add to the notational report 34, but no change may be made to that portion of the report that is generated by the program from the processed data. The charts 35 and 36 show the actual scan tracks relative to the length of the spine from the T-1 to the L-5 vertebrae. These charts provide a graphic representation of the subject's spine from scans taken while standing 35 and while bent at the waist 36 and show the distance from the T-1 to the L-5 vertebrae and the deviation to either side of the ideal straight spine.

Because the scans are automatically saved after successful processing, they can readily be retrieved and compared to view a history of the subject's spine and to determine if there has been any progression or regression of any curvature previously detected.

The scans are conducted in two positions, standing and bent at the waist, because these are the traditional positions for visually examining a subject for scoliosis. With the apparatus and method of the present invention, the bent at the waist position may be replaced with a second standing scan with the data from the two standing scans being processed and compared to generate the report. In that case, the scan report would include two charts 35 and 36 depicting the two standing scans and the notational report 34 would likewise be based on these two scans.

The foregoing presents preferred embodiments and a best mode of the subject invention. However, equivalent embodiments have been sufficiently alluded to and would be readily apparent to those of ordinary skill in the art and any such modifications may be made without departing from the scope and spirit of the present invention.

What is claimed is:

1. Apparatus for detection and measurement of spinal abnormalities comprising;
a hand held data input means having a track ball mechanism adapted for linear translation along a patient's spine and adapted to generate data signals corresponding to direction and distance of motion of said track ball,
a computer,
a data transmission connection between said hand held data input means and said computer adapted to transmit said data signals from said data input means to said computer, and
software running on said computer adapted to convert said data signals into a visual output representative of the patient's spine.

2. The apparatus of claim 1 wherein said hand held data input means comprises a housing having an upper surface and a lower surface, said upper surface configured to fit a user's palm and said lower surface of substantially planar dimension.

3. The apparatus of claim 2, wherein said track ball mechanism is contained within said housing and comprises a ball partially protruding from said lower surface of said housing, an X-axis roller and a Y-axis roller in contact with said ball and orthogonal to each other within said housing, a disk attached to each roller and rotatable therewith in response to motion of said ball, an X-axis detector and a Y-axis detector, and an electronic controller means, said disks having means cooperable with said X-axis detector and said Y-axis detector wherein said X-axis detector and said Y-axis detector generate electronic pulses in response to rotation of said disks, said pulses being received by said electronic controller means and formatted for transmission by said data transmission connection to said computer.

4. The apparatus of claim 3, further comprising a pair of parallel, spaced apart guide members extending from and perpendicular to said lower surface of said housing.

5. The apparatus of claim 4, wherein said guide members are linearly displaced from said partially protruding ball.

6. The apparatus of claim 4, wherein said guide members partially flank said ball.

7. The apparatus of claim 4 further comprising a light source located at a forward surface of said housing at an acute angle relative to said lower surface of said housing, whereby said light source provides an illumination spot guide ahead of said hand held data input means.

8. The apparatus of claim 7, wherein said light source is a low power laser pointer positions at an angle of from 30° to 50° relative to the plane of the lower surface of said housing.

9. The apparatus of claim 8 further comprising at least one control switch on said upper surface of said housing, said switch being manipulatable during use of said data input means, said switch being electrically connected to said electronic controller means whereby said data input means is activated or deactivated.

10. The apparatus of claim 8 further comprising an attitude indicator means on said upper surface of said housing, said attitude indicator means indicating deviation of said hand held data input means from a horizontal or vertical position relative to said spine.

11. The apparatus of claim 10 wherein said attitude indicator means comprises a bubble level.

12. The apparatus of claim 5 wherein said guide members comprise elongated skid bodies.

13. An apparatus for detection and measurement of spinal abnormalities comprising: a hand held data input means having a track ball mechanism adapted for linear translation along a patient's spine and adapted to generate data signals corresponding to direction and distance of motion of said track ball mechanism, said hand held data input means comprising a housing having an upper surface contoured to fit a human hand and a lower surface of substantially planar dimension, said housing containing a track ball mechanism comprising a ball rotatably confined therein so as to partially protrude from an aperture in said lower surface whereby said ball is capable of contacting and rolling along a surface with said housing, an X-axis roller and a Y-axis roller in contact with said ball within said housing and orthognal to each other, an X-axis pulse generator activated by said X-axis roller and a Y-axis pulse generator activatd by said Y-axis roller, said X-axis pulse generator and said Y-axis pulse generator each generating electronic signals in response to movement of said X-axis roller and said Y-axis roller by said ball, and an electronic controller means receiving said signals and formatting said signals into coordinate data, said housing further comprising a guide means on said lower surface in substantially linear relationship with said ball and a longitudinal axis of said housing, said guide means adapted to engage on either side of the spine and thereby guide said hand held data input means along said spine, and at least one control switch on said upper surface of said housing, said switch electrically connected to said electronic controller and actuatable during use of said hand held data input means to activate said controller and said pulse generators.

14. The apparatus of claim 13 further comprising data transmission means whereby said coordinate data is transmitted from said controller to a computer.

15. The apparatus of claim 14 further comprising a computer and a software program loaded on said computer whereby said computer receives said coordinate data from said controller and enters said data into said software program and whereby said software program processes said data and generates a visual representation of said spine on the basis of said data, said software program further having means to calculate a Cobb angle from said data and generate a report comprising said visual representation and said Cobb angle.

16. A method for scanning a spine, determining the presence of scoliosis and measuring deviation thereof comprising:
a) providing a hand held data input means and a computer, said hand held data input means comprising a track ball mechanism adapted for linear translation along a patient's spine, said track ball mechanism having means for generating data signals corresponding to direction and distance of motion of said track ball as X and Y coordinates and transmitting said data signals to said computer and control means for activating said hand held data input means, a software program loaded on said computer and adapted to receive said data signals, process said data signals and generate a visual representation of said spine based on said data signals, said software program including means to enter personal data, create a file for maintaining said data and said visual representation as a data base file, calculate a Cobb angle from said data, and generate a report thereof,
a) activating said software program and entering personal data for a patient,
b) conducting a first scan by placing said hand held data input means on said patient's back such that said track ball is in contact with said back and against said spine at or about the T-1 vertebra, actuating said control means on said hand held data input means and moving said hand held data input means along said spine so that said track ball rolls along and follows linear and lateral contours of said spine down to at or about the L-5 vertebra, whereby data signals are generated by said hand held data input means and are transmitted to said computer as a scan of said spine
c) recording and processing said signals on said computer, said processing of said signals comprising generating a visual representation of said spine and calculating a Cobb angle therefor and generating a report, and
e) diagnosing said patient based on analysis of said report.

17. The method of claim 16 further comprising conducting a second scan of said spine immediately after said first scan and recording and processing said signals of both scans on said computer, said processing of said signals comprising said software program aligning said scans to compensate for differences in actual starting location, conducting a compare tolerance routine and calculating a Cobb angle for both scans.

18. The method of claim 16 further comprising conducting a subsequent scan of the patient's spine at a later date and comparing the reports thereof for changes in condition of said spine.
